(19) United States
(12) Patent Application Publication
(10) Pub. No.: US 2014/0135697 A1
(43) Pub. Date: May 15, 2014

(54) DISPOSABLE HEAT EXCHANGE CASSETTE AND AN ASSEMBLY FOR HEATING INTRAVENOUS FLUID

(71) Applicant: Jan Erik Vest Hansen, Aarhus C (DK)

(72) Inventor: Jan Erik Vest Hansen, Aarhus C (DK)

(73) Assignee: VEINUX APS, Aarhus N (DK)

(21) Appl. No.: 14/127,407

(22) PCT Filed: Jun. 12, 2012
(86) PCT No.: PCT/DK2012/050199

§ 371 Date: Dec. 18, 2013

Related U.S. Application Data
(60) Provisional application No. 61/499,051, filed on Jun. 20, 2011.

(51) Int. Cl. A61M 5/44

(52) U.S. Cl. CPC A61M 5/44
USPC 604/113; 137/340

(57) ABSTRACT

A disposable cassette (1) is disclosed having an inlet port (4), an outlet port (5), and a heat exchanger element (3) with a fluid flow channel (9, 10, 15, 29) defined therein, the element being configured for conductive heat transfer between an exterior source and liquid in the fluid flow channel through at least one wall member of the heat exchanger element. The average cross sectional area of the outlet part of the fluid flow channel is substantially smaller than the average cross sectional area of the inlet part of the fluid flow channel. Furthermore is disclosed an assembly for connecting a source of intravenous fluid to a patient, the assembly comprising a sealed sterile packaging enclosing the disposable cassette, a first tube for connecting said source of intravenous fluid to the inlet port of the disposable cassette, and a second tube for connecting the outlet port of the disposable cassette to an intravenous catheter.

### FIELD OF INVENTION

The present invention relates to a disposable heat exchange cassette suitable e.g. for heating intravenous fluid before entering it into a patient as well as an assembly for heating intravenous fluid. However, the cassette is also suitable for cooling of e.g. intravenous fluid or for cooling of a blood stream before entering it into the patient.

### BACKGROUND

Disposable heat exchange cassettes e.g. for heating of intravenous fluid prior to entering the fluid into the vein of a patient are well-known in the art, such as from the US patent application No. US 2003/225396 A1 (Cartledge et al.) which also discloses a pump in the unit comprising the cassette for pumping the intravenous fluid. Other such cassettes are known from e.g. the U.S. Pat. No. 6,236,809 (Cassidy et al.), the international patent application No. WO 2006 /101743 (Smisson-Cartledge) and the US patent application No. US 2003/114795 (Medical Solutions).

A disposable heat exchange cassette is disclosed in the international patent application No. WO 01/64146 (Radiant Medical) where it is applied to heat a fluid that is used in an intravascular heat exchange catheter.

It is a general object of the present invention with respect to the cassettes discussed above to improve the performance of the cassette during use thereof.

### BRIEF DESCRIPTION OF THE PRESENT INVENTION

The present invention relates to such heat exchange cassette, where the fluid flow channel inside is substantially more narrow at the outlet part than at the inlet part, which solves a number of drawbacks of the known cassette design, where the fluid flow channel is of a substantially constant cross-sectional area throughout the full length of the fluid flow channel.

The increase in flow velocity of the fluid in the outlet part of the fluid flow channel as compared to the flow velocity at the inlet part of the fluid flow channel provides an improved local heat transfer coefficient between the wall member of the cassette which the fluid flows along and which is heated (alternatively cooled) from the outside of the cassette and thus at least partly compensates for the reduction of heat transfer caused by a lower temperature difference between the exterior source, which preferably is a heating source but alternatively may be a cooling source, on the opposite side of said wall member and the fluid at the outlet part of the fluid flow channel as compared to the fluid at the inlet part of the fluid flow channel. The local heat transfer coefficient is increased by the increase in flow velocity due to the decrease of thickness of the thermal boundary layer as well as the increased occurrence of turbulent flow structures in the fluid flow. The heat transfer at the outlet part of the fluid flow channel may also be increased by increasing the temperature of the exterior heat source at the part of the wall member along which the outlet part of the fluid flow channel extents and that may be combined with the present invention. However, without the increase in flow velocity caused by the narrowing of the cross sectional area of the fluid flow channel at the outlet part of the channel, the temperature of the exterior heat source should be raised more in order to provide the requested effect with an increased risk of local overheating of a part of the fluid flowing through the channel which is disadvantageous for the quality of at least some of the types of fluids applied as well as an increased risk of temperature overshoot during start and stop of the flow through the cassette. Thus, the increase in flow velocity at the outlet part of the fluid flow channel provides the advantage of improved heat transfer between the exterior heat source and the liquid in the outlet part of the channel without the above-mentioned drawbacks, although a moderate higher temperature of the exterior heat source at the outlet part as compared to the temperature at the inlet part may advantageously be combined with the present invention.

By improving the heat transfer to the liquid inside the fluid flow channel, the flow rate of the liquid through a cassette of a given size may be increased or the size of the cassette may be reduced for a given desired flow rate to be heated.

Another advantage of increasing the flow velocity at the outlet part of the fluid flow channel is that measurement of liquid temperature at the outlet part will be more precise due to the decreased thermal boundary layer. The liquid temperature at the outlet part of the heat exchanging fluid flow channel is a very important control parameter as it is the goal for the heating process to achieve a desired temperature of the liquid before it is entered into the vein of the patient.

The above advantages of having an increased flow velocity at the outlet part of the fluid flow channel is achieved with a minimized overall pressure loss by limiting the extend of the narrow part of the fluid flow channel to the outlet part and having a wider part of the fluid flow channel and thus a lower flow velocity and lower pressure loss at the inlet part of the flow channel.

Thus, the present invention relates to a disposable cassette having an inlet port, an outlet port, and a heat exchanger element with a fluid flow channel defined therein, the fluid flow channel having an inlet part in fluid connection with the inlet port and an outlet part in fluid connection with the outlet port, the heat exchanger element being configured for conductive heat transfer between an exterior heat source and liquid in the fluid flow channel through at least one wall member of the heat exchanger element, wherein the average cross sectional area of the outlet part of the fluid flow channel is substantially smaller than the average cross sectional area of the inlet part of the fluid flow channel.

In the present application, the cross sectional area of the fluid flow channel is defined as the area of the opening of the channel in a plane extending perpendicularly to the general flow direction of the fluid flow provided in the channel during use of the cassette.

In a preferred embodiment, the average cross sectional area of the outlet part of the fluid flow channel is less than 75%, preferably less than 65% of the average cross sectional area of the inlet part of the fluid flow channel. It is particularly preferred that the average cross sectional area of the outlet part of the fluid flow channel is in the range of 25% to 75% of the average cross sectional area of the inlet part of the fluid flow channel, preferably in the range of 35% to 65% thereof.

The inlet part of the fluid flow channel extends preferably at least 10%, particularly preferable at least 25% of the full length of the fluid flow channel. It is particularly preferred that the inlet part of the fluid flow channel extends in the range of 10% to 60% of the full length of the fluid flow channel, preferably in the range of 25% to 50% thereof.

By the term “the full length of the fluid flow channel” is herein understood the average distance the fluid will flow inside the fluid flow channel from the fluid enters the channel and until it leaves the channel.

The outlet part of the fluid flow channel extents preferably at least 10%, preferably at least 25% of the full length of the fluid flow channel. It is particularly preferred that the outlet part of the fluid flow channel extends in the range of 10% to 60% of the full length of the fluid flow channel, preferably in the range of 25% to 50% thereof.

The conductive heat transfer between an exterior heat source and liquid in the fluid flow channel through may during use of the cassette take place through one wall member of the heat exchanger element, but it is preferred that the cassette is formed as a flat part where the fluid flow channel is formed between two substantially parallel wall members and that the heat transfer takes place through both wall members, whereby the temperature difference between the fluid and the exterior heat sources may be lower and the risk of local overheating of the fluid will be reduced.

In a further preferred embodiment of the cassette according to the present invention, the heat exchanger element further comprises an intermediate part of the fluid flow channel In particular, the average cross sectional area of the intermediate part of the fluid flow channel is preferably in the range of 50% to 90% of the average cross sectional area of the inlet part of the fluid flow channel, more preferred in the range of 65% to 85% thereof.

The intermediate part of the fluid flow channel extends preferably at least 10%, and more preferably at least 25% of the full length of the fluid flow channel. In a particularly preferred embodiment, the intermediate part of the fluid flow channel extends in the range of 10% to 60% of the full length of the fluid flow channel, preferably in the range of 25% to 50% thereof.

In a further preferred embodiment of the cassette according to the present invention, a wall member of the heat exchanger element is provided with an indentation to accommodate a temperature sensor being external to the cassette, the indentation extending into the outlet part of the fluid flow channel. Thereby, the external temperature sensor is positioned to obtain a reliable measure of the fluid temperature at the outlet part of the fluid flow channel taking most advantage of the increased flow velocity at the outlet part and of the consequently thinner thermal boundary layer of the at the outlet part as discussed above. Correspondingly, it is preferred that a wall member of the heat exchanger element is provided with an indentation to accommodate a temperature sensor being external to the cassette, the indentation extending into the inlet part of the fluid flow channel.

The height of the fluid flow channel in a direction perpendicularly to said wall member of the heat exchanger element is preferably constant substantially throughout the full length of the fluid flow channel, and that height is in preferred embodiments of the present invention within the range of 0.6 to 2 millimetres, preferably within the range of 0.8 to 1.5 millimetres.

As an alternative, the varying cross-sectional area of the opening of the fluid flow channel is fully or partially obtained by varying the height of the fluid flow channel instead of or in combination with varying the width of said channel.

The fluid flow channel extents preferably along said wall member of the heat exchanger element over an area in the range of 30 to 85 cm², preferably in the range of 40 to 75 cm² and most preferred in the range of 50 to 65 cm².

In yet a preferred embodiment of the present invention, the cassette is further provided with a pump for pumping a flow of liquid from the inlet port to the outlet port via the fluid flow channel. It is preferred that the pump comprises two one-way valves and a length of an elastic tube extending there between so that the fluid flow may be driven by alternating compression and release of the length of the elastic tube by means of external pump drive means.

The present invention further relates to an assembly for connecting a source of intravenous fluid to a patient, the assembly comprising a sealed sterile packaging enclosing a disposable cassette according to the invention as disclosed above, a first tube having a liquid inlet for connecting to said source of intravenous fluid and a liquid outlet connected to the inlet port of the disposable cassette, and a second tube having a liquid inlet connected to the outlet port of the disposable cassette and a liquid outlet for connecting to an intravenous catheter.

Although the heat exchange cassette and the assembly according to the present invention mainly is described for being used for heating of an intravenous fluid, the cassette is also suitable for cooling of e.g. intravenous fluid or for cooling of a blood stream before entering it into the patient, which is used in therapeutic induced hypothermia, i.e. a reduction of the patient's body temperature in order to reduce the risk of the ischemic injury to tissue following a period of insufficient blood flow. Periods of insufficient blood flow may be due to cardiac arrest or the occlusion of an artery by an embolism, as occurs in the case of strokes.

### BRIEF DESCRIPTION OF THE DRAWINGS

A preferred embodiment of the present invention is disclosed in the attached drawing of which

FIG. 1 shows an assembled cassette according to an embodiment of the present invention,

FIG. 2 shows the pump included in the cassette, FIG. 3 shows the lower part of the pump head of the cassette,

FIG. 4 shows the lower part of the heat exchanger element of the cassette, and

FIG. 5 is an exploded view in perspective of the cassette.

### DETAILED DESCRIPTION OF THE PRESENT INVENTION

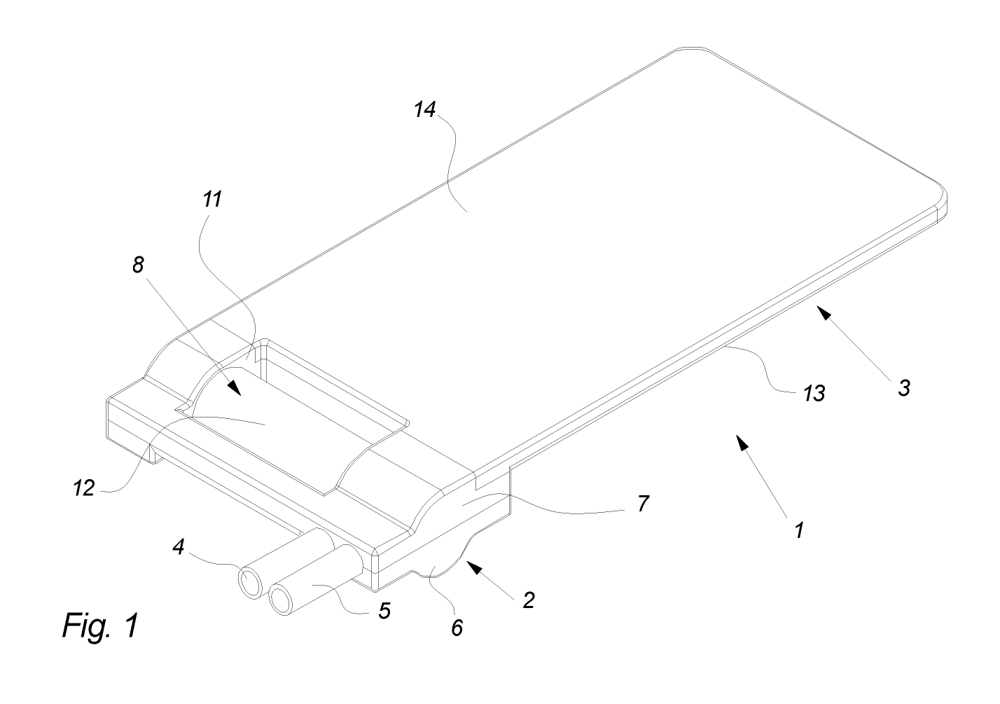

The disposable cassette 1 according to an embodiment of the present invention is shown assembled in FIG. 1 and comprises a pump head 2 and a heat exchanger element 3 build together as a single element. The exchange of fluid with the surroundings takes place through an inlet port 4 and an outlet port 5 arranged in the pump head 2.

The pump head 2 comprises a lower part 6 and an upper part 7 which, when the cassette 1 is assembled, together form a fluid tight enclosure for the pump 8 and connects the pump 8 to the inlet port 4 as well as to the inlet part 9 of the heat exchanger element 3 and which also connects the outlet part 10 of the heat exchanger element 3 to the outlet port 5. The upper part 7 of the pump head 2 has an opening 11 defined therein for allowing an external pump driver to have access to the elastic tube 12 of the pump 8. The heat exchanger element 3 is constituted by a lower part 13 and an upper part 14 which when the cassette 1 is assembled together form between the two and fluid flow channel 15 of which the inlet part 9 is in fluid contact with the inlet port 4 through the pump 8 and the outlet part 10 is in fluid contact with the outlet port 5.

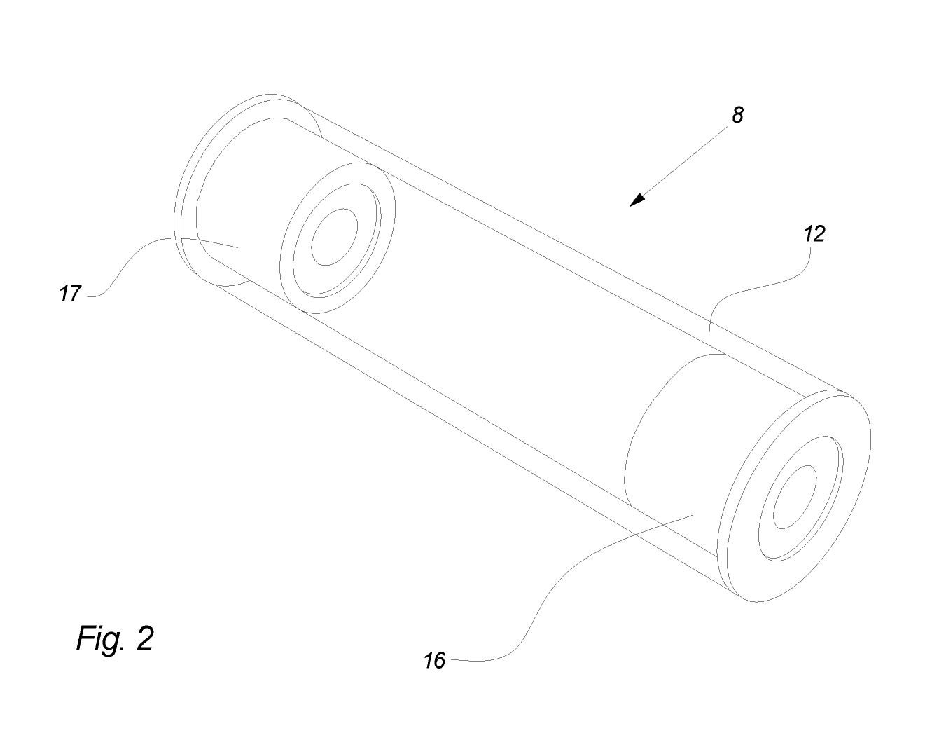

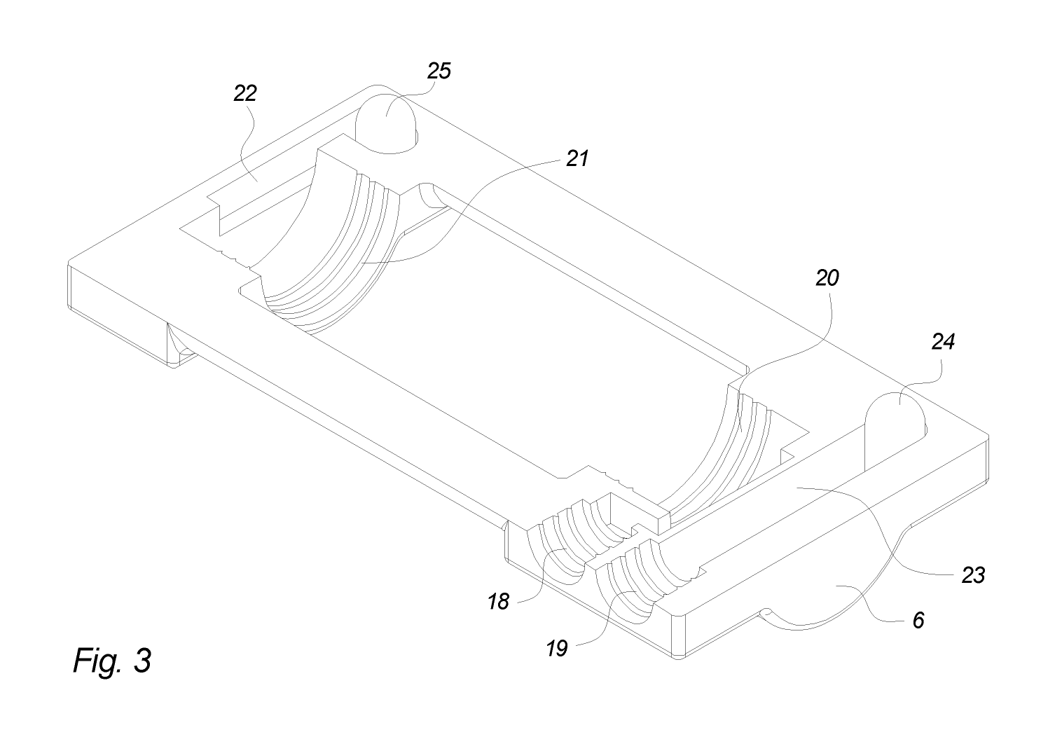

The pump 8 which is shown in FIG. 2 comprises a length of an elastic tube 12 and two one-way valves 16, 17 which are arranged in the tube 12 as inlet valve 16 and outlet valve 17, respectively. The pump 8 is arranged to establish and control the volumetric flow of fluid through the cassette 1 and is operated by means of a single piston externally to the cassette 1 that during operation alternately compresses and releases the compress of the length of elastic tube 12 between the two valves 16, 17.

The lower part 6 of the pump head 2 is shown in detail in FIG. 3 and comprises inlet and outlet areas 18, 19 for accommodating the inlet port 4 and the outlet port 5, respectively, two seats 20, 21 for supporting the ends of the pump 8, an inlet connecting passage 22 for providing the fluid connection between the outlet of the pump 8 and the inlet part 9 of the fluid flow channel 15 and an outlet connecting passage 23 for providing the fluid connection between the outlet part 10 of the fluid flow channel 15 and the outlet port 5.

The lower part 6 of the pump head 2 is provided at the outlet connecting passage 23 an indentation 24 which, when the cassette 1 is assembled, will extend into the outlet part 10 of the fluid flow channel 15, and the indentation 24 is provided with an opening (not visible in the drawings) so that an external temperature sensor (not shown) can be accommodated in the opening as discussed above for measuring the outlet fluid temperature. The inlet connecting passage 23 is in the same manner provided with a second indentation 25 that will extend into the inlet part 9 of the fluid flow channel 15 for accommodating another external temperature sensor for measuring the inlet fluid temperature. The first and the second indentation 24, 25 may be manufactured in a soft plastic material, such as a soft Poly Vinyl Chloride (PVC) or a material as soft polyethylene.

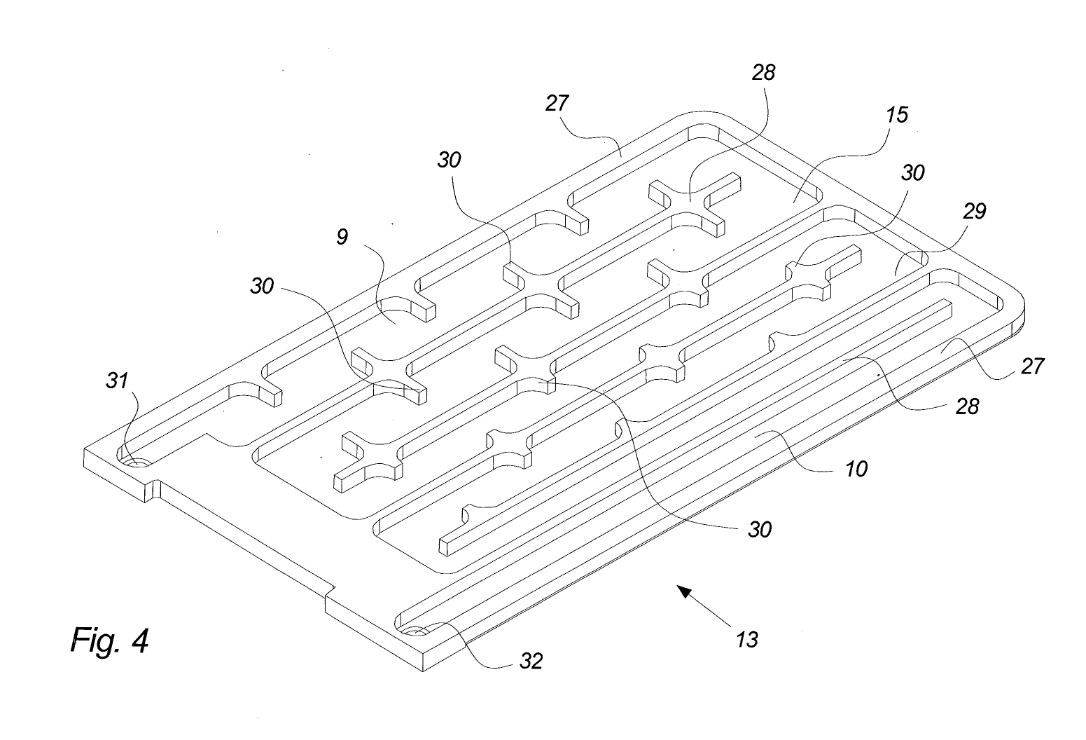

The lower part 13 of the heat exchanger element 3 is shown in a perspective view in FIG. 4. The part 13 is moulded in polyethylene and defines therein a fluid flow channel 15 which will closed by the upper part 14 of the heat exchanger element 3 when the cassette 1 is assembled. The part 13 comprises a wall member 26 of a thickness in the range of 0.3 to 0.75 millimetres, preferably of 0.5 millimetres through which the conductive heat transfer between an exterior heat source and liquid present in the fluid flow channel takes place during use of the cassette 1. The upper part 14 of the heat exchanger element has a corresponding thickness and may in the shown embodiment of the cassette 1 according to the invention also be used for conductive heat transfer from a second exterior heat source. Upstanding from the wall member 26 are a rim 27 and partition walls 28 which define the fluid flow channel 15 on the wall member 26 in three parts: an inlet part 9, an outlet part 10 and an intermediate part 29 of the fluid flow channel 15. The partition walls 28 are equipped with extensions 30 into the inlet part 9 and the intermediate part 29 of the fluid flow channel 15 so as to cause flow distortions and thereby improve the mixing of the liquid flowing through the channel 15 and consequently increase the heat transfer coefficient from the exterior heat source through the wall member 26 and to the liquid. The length of the three parts 9, 10, 29 of the fluid flow channel 15 are the same, 70 millimetres each, and the height is constant of 1 millimetre, whereas the width of the channel varies from 8 millimetres at the inlet part 9 to 6 millimetres at the intermediate part 29 and 4 millimetres at the outlet part 10 of the fluid flow channel 15. The inlet part 9 of the fluid flow channel 15 is equipped with an inlet opening 31 through which liquid will flow into the fluid flow channel 15 from the inlet connecting passage 22 when the cassette 1 is assembled and in use and the outlet part 10 is correspondingly equipped with an outlet opening 32 through which liquid will flow out from the fluid flow channel 15 into the outlet connecting passage 23 when the cassette 1 is assembled and in use.

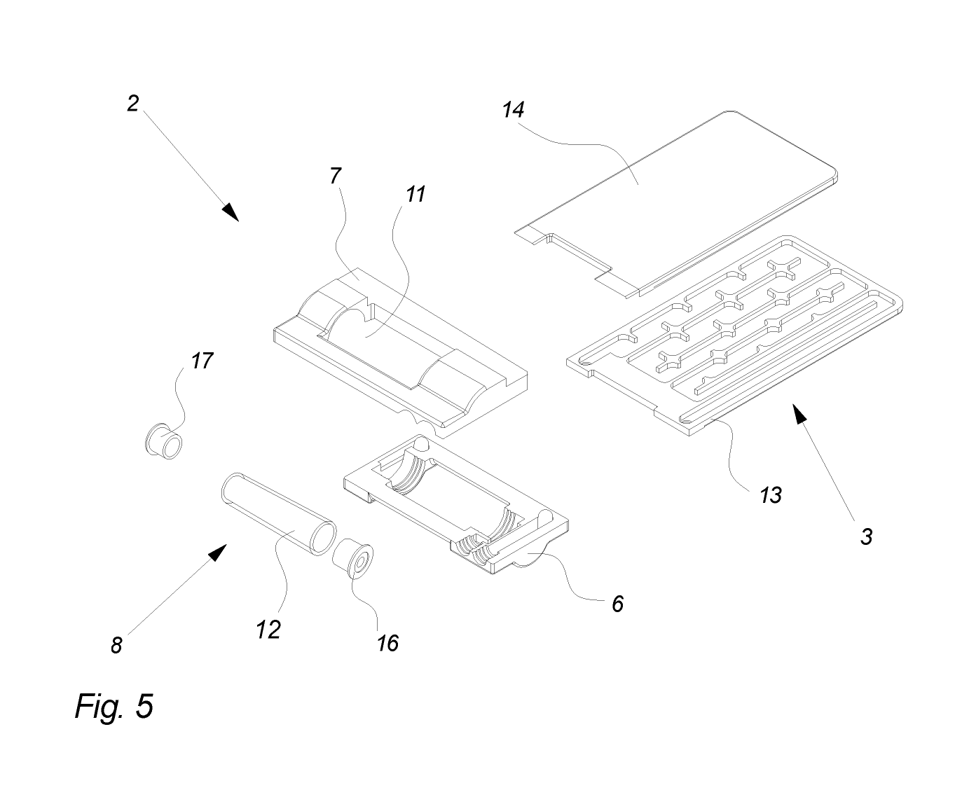

An exploded view of the cassette 1 assembly is shown in FIG. 5.

The four parts 6, 7, 13, 14 that form the outer housing of the cassette 1 are all moulded in polyethylene, but other suitable materials could be selected by the skilled person.

The exterior heat source may be divided into three heat exchange zones each extending along one of the three parts 9, 10, 29 of the fluid flow channel 15 so that the temperature of the heat source or cooling source is different for each part 9, 10, 29.

The cassette 1 may furthermore be equipped with a first tube having a liquid inlet for connecting to said source of intravenous fluid and a liquid outlet connected to the inlet port 4 of the disposable cassette 1 and a second tube having a liquid inlet connected to the outlet port 5 and a liquid outlet for connecting to an intravenous catheter. This assembly may be comprised in a sealed sterile packaging as a ready-for-use disposable equipment to be employed together with a reusable apparatus comprising the external temperature sensors and the exterior heat source as well as the external pump driver to establish and control the volumetric flow of fluid through the cassette 1 and by means of a single piston that during operation alternately compresses and releases the compress of the length of elastic tube 12 of the pump 8.

### LIST OF REFERENCE NUMERALS

1 Disposable cassette

2 Pump head

3 Heat exchanger element

4 Inlet port

5 Outlet port

6 Lower part of pump head

7 Upper part of pump head

8 Pump

9 Inlet part of the fluid flow channel

10 Outlet part of the fluid flow channel

11 Opening in upper part of pump head

12 Elastic tube of pump

13 Lower part of heat exchanger element

14 Upper part of heat exchanger element

15 Fluid flow channel in heat exchanger element

16 Inlet one-way valve of pump

17 Outlet one-way valve of pump

18 Inlet area

19 Outlet area

20, 21 Two seats for supporting ends of pump

22 Inlet connecting passage

23 Outlet connecting passage

24 Indentation

25 Second indentation

26 Wall member of lower part of heat exchanger element

27 Rim

28 Partition walls

29 Intermediate part of fluid flow channel

30 Extensions on partition walls

31 Inlet opening of fluid flow channel

32 Outlet opening of fluid flow channel

1. A disposable cassette comprising:
an inlet port,
an outlet port, and
a heat exchanger element with a fluid flow channel defined therein, the fluid flow channel having an inlet part in fluid connection with the inlet port and an outlet part in fluid connection with the outlet port,
the heat exchanger element being configured for conductive heat transfer between an exterior source and liquid in the fluid flow channel through at least one wall member of the heat exchanger element,
wherein an average cross sectional area of the outlet part of the fluid flow channel is substantially smaller than an average cross sectional area of the inlet part of the fluid flow channel.

2. A cassette according to claim 1, wherein the average cross sectional area of the outlet part of the fluid flow channel is less than 75% of the average cross sectional area of the inlet part of the fluid flow channel.

3. A cassette according to claim 2, wherein the average cross sectional area of the outlet part of the fluid flow channel is in the range of 25% to 75% of the average cross sectional area of the inlet part of the fluid flow channel.

4. A cassette according to claim 1, wherein said inlet part of the fluid flow channel extends at least 10% of the full length of the fluid flow channel.

5. A cassette according to claim 4, wherein said inlet part of the fluid flow channel extends in the range of 10% to 60% of the full length of the fluid flow channel.

6. A cassette according to claim 1, wherein said outlet part of the fluid flow channel extents at least 10% of the full length of the fluid flow channel.

7. A cassette according to claim 6, wherein said outlet part of the fluid flow channel extends in the range of 10% to 60% of the full length of the fluid flow channel.

8. A cassette according to claim 1, further comprising an intermediate part of the fluid flow channel.

9. A cassette according to claim 8, wherein the average cross sectional area of the intermediate part of the fluid flow channel is in the range of 50% to 90% of the average cross sectional area of the inlet part of the fluid flow channel.

10. A cassette according to claim 8, wherein said intermediate part of the fluid flow channel extends at least 10% of the full length of the fluid flow channel.

11. A cassette according to claim 10, wherein said intermediate part of the fluid flow channel extends in the range of 10% to 60% of the full length of the fluid flow channel.

12. A cassette according to claim 1, where a wall member of the heat exchanger element is provided with an indentation, to accommodate a temperature sensor being external to the cassette, the indentation extending into the outlet part of the fluid flow channel.

13. A cassette according to claim 1, where a wall member of the heat exchanger element is provided with an indentation to accommodate a temperature sensor being external to the cassette, the indentation extending into the inlet part of the fluid flow channel.

14. A cassette according to claim 1, wherein the height of the fluid flow channel in a direction perpendicularly to said wall member of the heat exchanger element is constant substantially throughout the full length of the fluid flow channel.

15. A cassette according to claim 14, wherein said height is within the range of 0.6 to 2 millimetres.

16. A cassette according to claim 1, wherein the fluid flow channel extents along said wall member of the heat exchanger element over an area in the range of 30 to 85 cm².

17. A cassette according to claim 1, which is further provided with a pump for pumping a flow of liquid from the inlet port to the outlet port via the fluid flow channel.

18. A cassette according to claim 17, wherein the pump comprises two one-way valves and a length of an elastic tube extending there between.

19. An assembly for connecting a source of intravenous fluid to a patient, the assembly comprising a sealed sterile packaging enclosing
a disposable cassette according to claim 1,
a first tube having a liquid inlet for connecting to said source of intravenous fluid and a liquid outlet connected to the inlet port of the disposable cassette, and
a second tube having a liquid inlet connected to the outlet port of the disposable cassette and a liquid outlet for connecting to an intravenous catheter.
